(19) United States
(12) Patent Application Publication
(10) Pub. No.: US 2009/0048461 A1
(43) Pub. Date: Feb. 19, 2009

(54) Regulators of Bacterial Signalling Pathways

(75) Inventor: Naresh Kumar, Maroubra (AU)

Correspondence Address: DARBY & DARBY P.C., P.O. BOX 770, Church Street Station, New York, NY, 10008-0770, US

(73) Assignee: Biosignal Limited, Eveleigh (AU)

(21) Appl. No.: 11/571,033

(22) PCT Filed: Jun. 21, 2005
(86) PCT No.: PCT/AU05/00881

§ 371 Date: Mar. 29, 2007

(30) Foreign Application Priority Data
Jun. 21, 2004 (AU) 2004903362

(51) Int. Cl. C07C 69/66; C07C 45/65

(52) U.S. Cl. 560/130; 560/174; 568/397

(57) ABSTRACT

The present invention provides a method for the preparation of compounds of formula (II). The invention also provides novel compounds of formula (II) and their use in medical, scientific and/or biological applications.

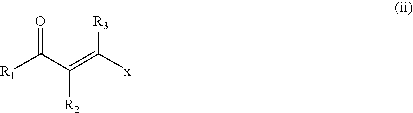

### FIELD OF THE INVENTION

The present invention relates to novel synthetic methods, to the products of such novel methods, and to uses of these products. In particular, the present invention provides methods for the decarboxylation of substituted or unsubstituted dibrominated 4-oxoalkanoic acids and relates to the products of such a method. The present invention also relates to novel compounds.

### BACKGROUND OF THE INVENTION

The family Bonnemaisoniaceae is widely distributed in both tropical and temperate waters and flourishes in areas containing high concentrations of herbivores. The members of this family (Asparagopsis, Delisea, Ptilonia, Leptophyllis, Bonnemaisonia) are generally unpalatable to herbivores and it has been shown that three of the more cosmopolitan genera (Delisea, Asparagopsis, and Bonnemaisonia) as well as the respective alternate heteromorphic tetrasporophyte phases for Asparagopsis, and Bonnemaisonia (Falkenbergia and Trailliella respectively) inhibit growth in vitro in a number of pathogens. These genera produce a rich variety of halogen containing compounds. For example Asparagopsis produces small, volatile polyhalogenated compounds; the genera, Bonnemaisonea, Delisea and Ptilonia, on the other hand, produce halogen containing compounds with C7 and C9 composition. These include fimbrolides, polyhalogenated 1-octen-3-ones, halomethanes, haloacetaldehydes, haloacetones, halobutenones, haloacctic and haloacrylic acids.

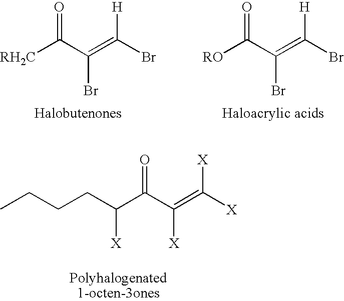

These compounds possess potent antimicrobial activity and act as antifeedants in nature. The small volatile compounds e.g. halomethanes, haloacetaldehydes and haloacetones are generally toxic and hence are hot suitable for any potential antimicrobial applications. The halobutenones, polyhalogenated 1-octene-3-one and the haloacrylic acids, on the other hand, have the potential to act as antibiotics themselves.

We have been engaged in the development of novel antimicrobials from the related red marine alga Delisea pulchra (see WO 96/29392 and WO 99/53915, the disclosures of which are incorporated herein in their entirety by cross-reference). In the course of this work we have developed a reaction which yields a variety of halomethylene substituted alkanones of formula II in high yields.

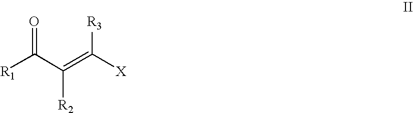

wherein R₁, R₂ and R₃, which may be the same or different, are independently selected from H, halogen, alkyl, alkoxy, alkenyl, alkynyl, aryl, arylalkyl, carboxyl, acyl, acyloxy, acylamino, formyl and cyano whether unsubstituted or substituted, optionally interrupted by one or more hetero atoms, straight chain or branched chain, hydrophilic, hydrophobic or fluorophilic and X is a halogen.

Halomethylene alkanones are analogues of the natural halobutenones where the halogen group alpha to the carbonyl has been replaced by an alkyl group. Furthermore halomethylene alkanones can also be considered as potential analogues of the natural halogenated 1-octen-3-ones where the dihalomethylene end group present in the natural compounds has been replaced by a halomethylene group and the bromine atom alpha to the carbonyl group has been replaced by an alkyl group.

In spite of their potent biological activity, very few compounds related to the parent structure of halobutenones, halogenated 1-octen-3-ones and haloacrylic acids have been reported in the literature. In particular, information regarding the brominated analogues of these compounds is rather scarce; the marine natural products are predominantly brominated. The bulk of the very few examples of the bromomethylene alkanone that have been reported in the literature contain a hydroxybenzyl substituent, and the antimicrobial activity of these compounds has not been investigated.

International Patent Publication Nos. WO 01/043739, WO 02/047681 and WO 02/102370 disclose the general structure of Formula II and that compounds of this structure may potentially have antibacterial properties. However, these publications do not disclose methods of preparing these compounds and, further, only exemplify one or two members haying the general structure of formula II.

As far as we are aware, there is not at present a general method suitable for the synthesis of these analogues. The few reported syntheses of these compounds utilise a modified Baylis-Hillman reaction of acetylenic ketones. This reaction, however, requires the use of a highly reactive aromatic aldehyde e.g. ρ-nitrobenzaldehyde thus limiting the scope of this reaction only to the synthesis of hydroxymethylphenyl substituted chloromethylene alkanones.

The halomethylene alkanones can be considered as key intermediates in the preparation of further analogues (if halobutenones and halogenated 1-octen-3-ones as the acetyl methyl and the allylic alkyl group present in the halomethylene alkanones should be able to be further functionalised by standard free radical halogenation and oxidation reactions.

We have found conditions that, surprisingly, enable the synthesis of halomethylene alkanones via the decarboxylation of 2,3-dihalo-4-oxoalkanoic acids under mild basic conditions. This i method is particularly useful hi the synthesis of bromethylene alkanones.

### SUMMARY OF THE INVENTION

Accordingly, in a first aspect, the present invention provides a method for the preparation of a compound of formula II

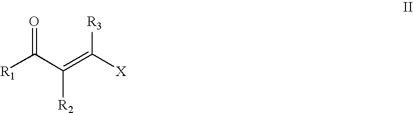

wherein R₁, R₂ and R₃, which may be the same or different, are independently selected from H, halogen, alkyl, alkoxy, alkenyl, alkynyl, aryl, arylalkyl, carboxyl, acyl, acyloxy, acylamino, formyl and cyano whether unsubstituted or substituted, optionally interrupted by one or more hetero atoms, straight chain or branched chain, hydrophilic, hydrophobic or fluorophilic and X is a halogen;
the method comprising decarboxylating a compound of formula I

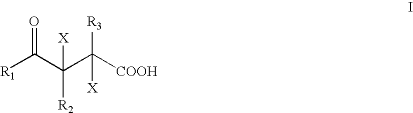

wherein R₁, R₂, R₃ and X are as defined above, and
wherein the decarboxylation is carried out in the presence of a mild base, optionally in the presence of a solvent.

In a second aspect, the present invention provides a compound of formula II produced by the method according to the first aspect of the present invention.

In a third aspect, the present invention provides a method of use of the compound of the second aspect in a medical, scientific and/or biological application.

In a fourth aspect, the present invention provides a compound of formula II

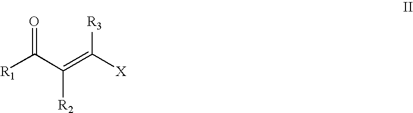

wherein R₁, R₂ and R₃, which may be the same or different, are independently selected from H, halogen, alkyl, alkoxy, alkenyl, alkynyl, aryl, arylalkyl, carboxyl, acyl, acyloxy, acylamino, formyl and cyano whether unsubstituted or substituted, optionally interrupted by one or more hetero atoms, straight chain or branched chain, hydrophilic, hydrophobic or fluorophilic and X is a halogen.

### BRIEF DESCRIPTION OF THE FIGURES

FIG. 1 shows the results of a beta-galactosidase assay of the prior art compound 3-(bromomethylene)-2-butanone (80).

FIG. 2 shows the results of a beta-galactosidase assay of 2-(bromomethylene)-3-pentanone (123).

FIG. 3 shows a graph depicting the effect of 3-(bromomethylene)2-hexanone (122) on the growth of Staphylococcus aureus. The absorbance is proportional to the number of bacteria.

FIG. 4 shows a graph depicting the effect of 2-(bromomethylene)-3-pentanone (123) on the growth of Staphylococcus aureus. The absorbance is proportional to the number of bacteria.

FIG. 5 shows the effect of 3-(bromomethylene)-2-hexanone (122) and 2-(bromomethylene)-3-pentanone (123) against attachment of Pseudomonas aeruginosa (PA01 DO)

FIG. 6 shows the effect of 3-(bromomethylene)-2-heptanone (101), 3-(bromomethylene)-2-hexanone (122) and 2-(bromomethylene)-3-pentanone (123) on the bioluminescence activity in Vibrio harveyi Al-2 assay.

FIG. 7 shows the effect of 2-(bromomethylene)-3-pentanone (123) and 3-bromomethylene)-2-tridecanone (compound 124) on the growth of Porphyromonas canoris.

FIG. 8 shows the effect of 2-(bromomethylene>3-pentanone (123) and 3-(bromomethylene>-2-tridecanone (compound 124) on the attachment of Porphyromonas canoris.

FIG. 9 shows the effect of 2-(bromomethylene)-3-pentanone (123) on the growth of Pseudomonas aeruginosa.

FIG. 10 shows the effect of 2-(bromomethylene)-3-pentanone (123) on the attachment of Pseudomonas aeruginosa.

### DETAILED DESCRIPTION OF THE INVENTION

In a first aspect, the present invention provides a method for the preparation of a compound of formula II

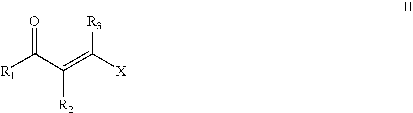

wherein R₁, R₂ and R₃, which may be the same or different, are independently selected from H, halogen, alkyl, alkoxy, alkenyl, alkynyl, aryl, arylalkyl, carboxyl, acyl, acyloxy, acylamino, formyl and cyano whether unsubstituted or substituted, optionally interrupted by one or more hetero atoms, straight chain or branched chain, hydrophilic, hydrophobic or fluorophilic and X is a halogen;
the method comprising decarboxylating a compound of formula I

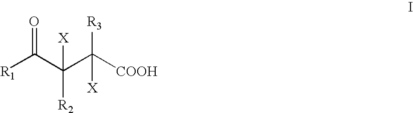

wherein R₁, R₂, R₃ and X are as defined above, and
wherein the decarboxylation is carried out in the presence of a mild base, optionally in the presence of a solvent.

In formula II, a particular geometry is not to be taken as specified. For example, the formula covers both E- and Z-isomers.

The substituents of starting compound of formula I and halomethylene alkanone or formula II are preferably as follows:

R₁, R₂ and R₃, which may be the same or different, are independently selected from H, halogen, alkyl, alkoxy, oxoalkyl, alkenyl, aryl or arylalkyl whether unsubstituted or substituted, optionally interrupted by one or more hetero atoms, straight chain or branched chain, hydrophilic, hydrophobic or fluorophilic;
X is a halogen;

More preferably, the starting compound of formula I and halomethylene alkanone of formula II comprise the following substituents:

R₁, R₂ and R₃ are independently H, halogen, alkyl, alkoxy, oxoalkyl, alkenyl, aryl or arylalkyl whether unsubstituted or substituted, optionally interrupted by one or more hetero atoms, straight chain or branched chain, hydrophilic, hydrophobic or fluorophilic; and

### X is Br, F or I;

Most preferably, the starting dihalo acid of formula I and halomethylene alkanone of formula II comprise the following substituents:

R₁, R₂ and R₃, which may be the same or different, are independently selected from H, halogen, alkyl, alkoxy, oxoalkyl, alkenyl, aryl or arylalkyl whether unsubstituted or substituted, optionally interrupted by one or more hetero atoms, straight chain or branched chain, hydrophilic, hydrophobic or fluorophilic; and

### X is a Br.

Preferably, at least one of R₁, R₂, R₃ is an alkyl group. Most preferably, at least one of R₁ and R₂ is alkyl and R₃ is H.

The method of the present invention has particular application in the decarboxylation of compounds of formula I wherein X is a bromine.

Preferably the compound of formula II produced by the method of present invention is selected from halomethylene alkanones.

The term “alkyl” as used herein is taken to mean both straight chain alkyl groups such as methyl, ethyl, propyl, isopropyl, butyl, isobutyl, sec-butyl, tertiary butyl, and the like. Preferably the alkyl group is a lower alkyl of 1 to 10 carbon atoms, preferably 1 to 6 carbon atoms. The alkyl group may optionally be substituted by one or more groups selected from alkyl, cycloalkyl, alkenyl, alkynyl, halo, haloalkyl, haloalkynyl, hydroxy, alkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocyclyl, heterocycloxy, heterocyclamino, haloheterocyclyl, alkylsulfenyl, alkylcarbonyloxy, alkylthio, acylthio, phosphorus-containing groups such as phosphono and phosphinyl.

The term “alkoxy” as used herein denotes straight chain or branched alkyloxy, preferably C₁₋₁₀ alkoxy. Examples include methoxy, ethoxy, n-propoxy, isopropoxy and the different butoxy isomers.

The term “alkenyl” as used herein denotes groups formed from straight chain, branched or mono- or polycyclic alkenes and polyene. Substituents include mono- or poly-unsaturated alkyl or cycloalkyl groups as previously defined, preferably C₂₋₁₀ alkenyl. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-deccuyl, 3-deccnyl, 1,3-butadienyl, 1-4,pentadienyl, 1,3-cyclopentadicuyl, 1,3-hexadienyl, 1,4-hexadienyl, ) ,3-cyclohexadienyl, 1,4-cyclohexadienyl. 1,3-cycloheptadienyl, 1,3,5-cycloheptatrienyl, or 1,3,5,7-cyclooctatetraenyl.

The term “halogen” as used herein denotes fluorine, chlorine, bromine or iodine, preferably bromine or fluorine.

The term “heteroatoms” as used herein denotes O, N or S.

The term “acyl” used either alone or in compound words such as “acyloxy”, “acylthio”, “acylamino” or “diacylamino” denotes an aliphatic acyl group and an acyl group containing a heterocyclic ring which is referred to as heterocyclic acyl, preferably a C₁₋₁₀ alkanoyl. Examples of acyl include carbamoyl; straight chain or branched alkanoyl, such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl; alkoxycarbonyl, such as methoxycarbonyl, ethoxycarbonyl, t-butoxycarbonyl, t-pentyloxycarbonyl or heplyloxycarbonyl; cycloalkanecarbonyl such as cyclopropanecarbonyl cyclobutanecarbonyl, cyclopentanecarbonyl or cyclohexanecarbonyl; alkenesulfonyl, such as methanesulfonyl or ethanesulfonyl; alkoxysulfonyl, such as methoxysulfonyl or ethoxysulfonyl; heterocycloalkanecarbouyl; heterocyclyoalkanoyl, such as pyrrolidinylacetyl, pyrrolidinylpropanoyl, pyrrolidinylbutanoyl, pyrrolidinylpentanoyl, pyrrolidinylhexanoyl or thiazolidinylacetyl; heterocyclylalkenoyl, such as heterocyclylpropenoyl, heterocyclylbutenoyl, heterocyclylpentenoyl or heterocyclylhexenoyl; or heterocyclylglyoxyloyl, such as, thiazolidinylglyoxyloyl or pyrrolidinylglyoxyloyl.

The term “alkynyl” as used herein, refers to straight chain or branched hydrocarbon groups containing one or more triple bonds. Suitable alkynyl groups include, hut are not limited to ethynyl, propynyl, butynyl, pentynyl and hexenyl.

The term “aryl” us used herein, refers to C₆-C₁₀ aromatic hydrocarbon group, for example phenyl or naphthyl.

The term “arylalkyl” includes, for example, benzyl.

The term “fluorophilic” is used to indicate the highly attractive interactions between certain groups, such as highly fluorinated alkyl groups of C₄-C₁₀ chain length, have for perfluoroalkanes and perfluoroalkane polymers.

The mild basic catalysts may be selected from catalysts that are insoluble in the reaction medium or catalysts that are soluble in the reaction medium. Examples of insoluble basic catalysts include basic resins, basic salts and basic polymers. Examples of soluble basic catalysts include triethylamine, pyridine, 1,4-diazabicyclo[2.2.2]octane (DABCO), 4-(dimethylamino)pyridine (DMAP), 1,8-diazabicyclo[5.4.0]undec-7-ene (DBU).

Preferably, decarboxylation is carried out using triethylamine or DBU by itself or mixed with another base. More preferably decarboxylation is carried out using triethylamine.

The decarboxylation may be performed with a mild base in the presence or absence of a solvent. The solvent may be any suitable solvent. Preferable solvents in the present invention include alkyl acetates, aromatic hydrocarbons, chlorinated alkanes, tetrahydrofuran, diethyl ether, and dioxane. More preferably, the solvents are alkyl acetates and chlorinated alkanes. Most preferably, the solvent is dichloromethane, as well as dichloroethane and trichloroethane.

The reaction is preferably carried out at mild temperatures. Preferably the reaction is performed at a temperature in the range of from about −20-150° C.

The reaction lime may range from about 2 hours to 12 hours or more and is typically about 2 hours or more. It will be appreciated that reaction conditions may be varied depending on the individual nature of the substrate and the desired rate of the reaction.

The brominated keto acids used in this invention can be obtained by the addition of bromine to the corresponding 4-oxo-2-alkenoic acids as described in our International Patent Application No. PCT/AU01/00781, published as WO02/00639, the disclosure of which is incorporated herein in its entirety by cross-reference.

The present inventors have found that with a judicious choice of base catalyst and solvent, the decarboxylation of brominated keto acids can be carried out with few side products and in high yields to the corresponding halomethylene alkanones. In particular the use of triethylamine in dichloromethane provided very efficient decarboxylation of 2,3-dibromoketo acids to bromomethylene alkanones.

The halomethylene alkanones described in this invention were found to be stable and no further reaction of the halomethylene alkanones was observed even if the reaction was continued for a longer period of time. This reaction appears to be quite general and was repeated on a several gram scale.

In a second aspect, the present invention provides a compounds of formula II produced by the method according to the first aspect of the present invention. Preferably the compound of formula II is a halomethylene alkanone.

In a third aspect, the present invention provides a methods of use of a compound of formula II in a medical, scientific and/or biological application.

In preferred forms of the third aspect, the medical, scientific and/or biological applications include use of the compounds of formula II in products selected from: cleaning agents in the home and industrial settings; antifouling paints, water treatment products; antibacterial agents in the treatment of mammals; antibacterial additives and preservatives in medical or surgical devices, disinfectants, soap formulations, shampoo formulations, hand wash formulations, dentrification formulations, detergents for laundry or dishes, wash and treatment solutions for topical use including those designed for treating contact lenses; instruments and devices including contact lenses; and other disinfecting and antibacterial formulations.

In a fourth aspect, the present invention provides a compound of formula II

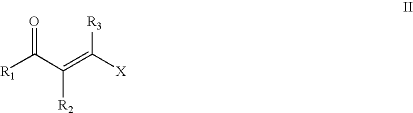

wherein R₁, R₂ and R₃ , which may be the same or different, are independently selected from H, halogen, alkyl, alkoxy, alkenyl, alkynyl, aryl, arylalkyl, carboxyl, acyl, acyloxy, acylamino, formyl and cyano whether unsubstituted or substituted, optionally interrupted by one or more hetero atoms, straight chain or branched chain, hydrophilic, hydrophobic or fluorophilic and X is a halogen.

In a preferred form, R₁ is alkyl; R₂ is alkyl or aryl; R₃ is H; X is Br or F.

More preferably, where R₁ is methyl, R₂ is not C₁₀alkyl, methyl, CH₂CH₂CO₂CH₃, CH₂CH₂NO₂ or CH₂CH₂CH₂OC(O)Ph.

Preferably, R₁ is C₂₋₁₀ alkyl. More preferably, R₁ is ethyl.

Preferably, X is Br.

The compounds of the Examples are preferred. Particularly preferred is the compound 2-(bromethylene)-3-pentanone (compound 123).

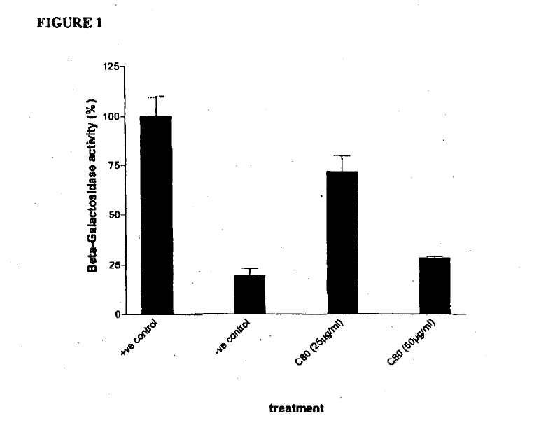

Of interest are (bromomethylene) alkanones as these compounds show important biological activities (see FIGS. 1-6). For example it has been found that

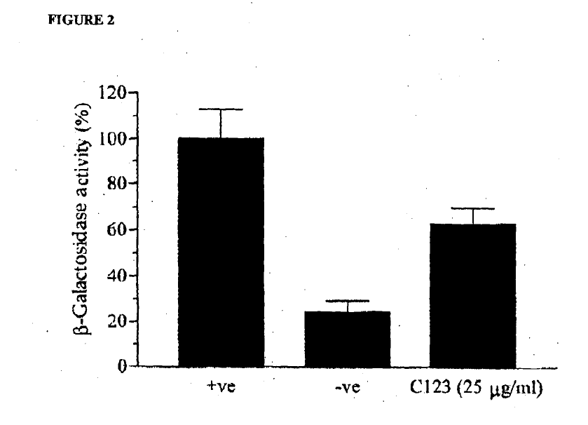

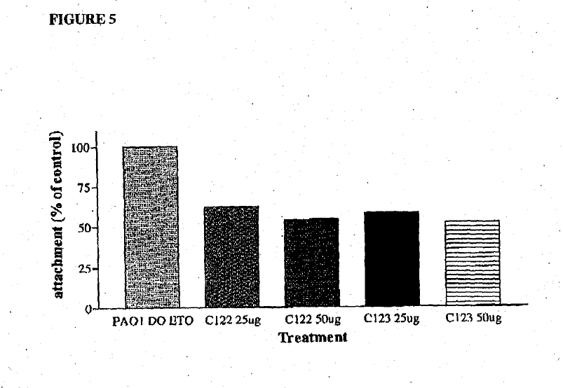

3-(bromomethylene)-2-butanone and 2-(bromomethylene)-3-pentanone act as inhibitors of two-component signal transduction systems (see FIGS. 1 and 2 which show the beta galactosidase activity). Furthermore 3-(bromomethyl)-2-hexauone and
2-(bromomethylene)-3-pentanone reduce the attachment of Pseudomonas aeruginosa (PA01 DO) (FIG. 5).

Further, (bromomethylene) alkanones are of particular interest as it has been shown that such compounds may have a negligible effect on the growth of bacteria while significantly limiting the attachment of the bacteria to surfaces. See, for instance, examples 9 and 10 where it is shown that compound 123 has a relatively insignificant effect on the growth of Pseudomonas aeruginosa and Porphyromonas canoris but significantly limits the attachment of these bacteria to a surface.

### EXAMPLES

The invention is further described in and illustrated by the following examples. The examples are not to be construed as limiting the invention in any way.

### Example 1

### General Method for the Synthesis of Dibromo Oxoalkanoic Acids

A solution of bromine (0.06 mol) in dry dichloromethane (8 ml) was added slowly to an ice-cooled solution of 4-oxo-2-alkenoic acid (0.03 mol) in dry dichloromethane (30 ml). The mixture was stirred in an ice-bath for 0.5 h and then at room temperature for 0.5 h. The resulting solution was washed with aqueous sodium metabisulfite (0.5 M, 30 ml) and brine (30 ml). The solution was dried over sodium sulfate and evaporated to dryness to yield the crude 2,3-dibromo-4-oxoalkanoic acid a pale brown oil (60-65%).

The crude product was used for the decarboxylation step without further purification.

### Example 2

### General Method for the Synthesis of 3-(Bromomethylene)-2-Alkanones and 2-Bromo-4-Oxo-2-Alkenoic Acids

A solution of triethylamine (8.7 mmol) in dichloromethane (5 ml) was added dropwise with stirring to an ice-cooled solution of the dibromo-4-oxoalkanoic acid (3.5 mmol) in dry dichloromethane (10 ml). The mixture was stirred in ice for 2 h and then at room temperature overnight. The resulting mixture was poured into dilute hydrochloric acid (50 ml, 2M) and extracted with dichloromethane (3×20 ml). The combined dichloromethane extracts were washed with brine (100 ml), dried over anhydrous sodium sulfate and evaporated to yield the 3-(bromomethylene)-2-alkanone as a light brown oil. The crude product was chromatographed on silica gel using dichloromethane as the eluent to yield the 3-(bromomethylene)-2-alkanone as a colourless oil (52-70%).

Further elution of the column with dichloromethane/ethyl acetate (3:1) yielded the 2-bromo-4-oxo-2-alkenoic acid (20-30%) as an oil which solidified on standing at room temperature.

### Example 3

The following examples of (bromomethylene)alkanones and 2-bromo-4-oxo-2-alkenoic acids were prepared using the general procedures described above.

### 3-(Bromomethylene)-2-Butanone (180)

Although compound 80 has previously been disclosed, to the applicant's knowledge, its synthesis has not been reported.

Yield 62%. vₘₐₓ 3090, 2820, 1670, 1595, 1425, 1360, 1300, 1220, 1095, 1010, 800 cm¹. ¹H n.m.r. δ (CDCl₃) 1.96, s, 3H, CH3; 2.33, s, 3H, COCH3; 7.49, s, 1H, 3-CHBr. ¹³C n.m.r. δ (CDCl₃): 14.7, C4; 25.9, Cl; 124.2, 3-CHBr; 143.2, C3; 195.2, C2. Mass spectrum: m/z 164 (M(⁸¹Br), 10%); 162 (M(79Br), 20%); 149 (20); 147 (20); 121 (10), 119 (10).

### 2-Bromo-3-Methyl-4-Oxo-2-Pentenoic Acid

Yield 27%. vₘₐₓ 3250, 2900, 2850, 1740, 1650, 1450, 1370, 1300, 1270, 1190, 1110, 1010, 900, 870, 800, 760 cm⁻¹. ¹H n.m.r. δ (CDCl3) 1.69, s, 3H, CH3; 2.08, s, 3H. Mass spectrum: m/z 208 (M(⁸¹Br), 10%); 206 (M(79Br), 10%); 193 (20); 191 (20); 163 (100); 165 (100).

### 3-(Bromomethylene)-2-Hexanone (Compound 122)

Yield 58%. vₘₐₓ 3090, 2950, 2850, 1670, 1590, 1460, 1420, 1360, 1310, 1210, 1120,1020, 1010, 940, 800. 740 cm−1. ¹H n.m.r. δ (CDCl3) 0.94, t, J 7.2 Hz, CH3; 1.42, m, 2H, Cl12; 2.28, s, 3H, COCH3; 2.43, t, J 7.2 Hz, CH2; 7.48, s, 1H, 3-CHBr. ¹³C n.m.r. δ (CDCl3): 14.0, C6; 21.0,C5; 26.2, Cl; 30.7, C4; 124.3, 3-CHBr; 147.5, C3; 195.2, C2. Mass spectrum: m/z 192 (M(⁸¹Br), 10%); 190 (M(⁷⁹Br), 10%); 177 (100); 175 (100); 149 (30), 147 (30), 121 (70), 119 (70), 111 (100), 93 (100).

### 3-(Bromomethylene)-2-Heptanone (Compound 101)

Yield 68%. vₘₐₓ 3090, 2959, 2860, 1679, 1595, 1465, 1364, 1308, 1206, 1118, 1015, 945, 800, 742 cm⁻¹. ¹H n.m.r. δ (CDCl₃) 0.91, t, J 6.4 Hz, CH3; 1.34, m, 4H, (CH2)2; 2.32, s, 3H, COCH3; 2.46.1, J 7.4 Hz, CH2; 7.46, s, 1H, 3-CHBr. ¹³C n.m.r. δ (CDCl₃): 13.7, C7; 22.6, C6; 26.1, Cl; 28.5, C3; 29.7, C4; 123.9, 3-CHBr; 147.6, C3; 195.1, C2. Mass spectrum: m/z 206(M(⁸¹Br), 5%); 204 (M(⁷⁹Br), 5%); 191 (100); 189 (100); 177 (30), 175 (30), 164 (40), 162 (40), 149 (100), 147 (100), 125 (70), 107 (70).

### 3-(Bromomethylene)-2-Nonanone

Yield 56%. vₘₐₓ 3090, 2928, 2858, 1680, 1595, 1458, 1364, 1312, 1220 cm⁻¹. ¹H n.m.r. δ (CDCl₃) 0.88, t, J 6.4 Hz, Cl13; 1.30, m, 4H, (CH2)4; 2.32, s, 3H, COCH3; 2.44, t, J 7.4 Hz, CH2; 7.45, s, 1H, 3-CHBr. 13C n.m.r. δ (CDCl₃): 13.9, C9; 22.4, C8; 26.2,Cl; 27.5,C4; 28.7, C6; 29.1, C7; 31.4, C5; 123.9,3-CHBr, 147.6, C3; 195.1, C2. Mass spectrum: m/z 234 (M (⁸¹Br), 5%); 232 (M(⁷⁹Br), 5%); 219 (25); 217 (25); 177 (15), 175 (15), 164 (40), 162 (40), 149 (100), 147 (100), 135 (100), 107 (70).

### 3-(Bromomethylene)-2-Decanone

Yield 71%. vₘₐₓ 3090, 2926, 2856, 1680, 1594, 1465, 1432, 1363, 1301, 1220, 1127, 1055, 1028, 950, 802, 742 cm⁻¹. ¹H n.m.r. δ (CDCl₃) 0.88, t, J 6.4 Hz, CH3; 1.30, m, 4H, (CH2)5; 2.31,s, 3H, COCH3; 2.45, t, J 7.2 Hz, CH2; 7.45,s, 1H, 3-CHBr. ¹³C n.m.r. δ (CDCl₃): 14.1, C10; 22.6, C9; 26.3, Cl; 27.7, C4; 28.8. C5; 29.0, C6; 29.6, C7; 31.8, C8; 124.1, 3-CHBr; 147.7, C3; 195.2, C2. Mass spectrum: m/z 249 (M(⁸¹Br), 5%); 247 (M(⁷⁹Br), 5%); 233 (20); 231 (20); 167 (100), 149 (100), 147 (100), 123 (80), 109 (100).

### 3-(Bromomethylene)-2-Tridecanone (Compound 124)

Although compound 124 has previously been disclosed, to the applicant's knowledge, its synthesis has not been reported.

Yield 52%. vₘₐₓ 3090, 2925, 2854, 1680, 1594, 1465, 1363, 1297, 1217, 1127, 1045, 951, 802, 742 cm⁻¹.¹H n.m.r. δ (CDCl₃) 0.88, t, J 6.4 Hz, CH3; 1.26, m, 4H, (CH2) 8; 2.32, s, 3H, COCH3; 2.45, t, J 6.8 Hz, CH2; 7.45, s, 1H, 3-CHBr. ¹³C n.m.r. δ (CDCl₃): 14.5, C13; 23.1 C12; 26.6, CH3; 28.0, CH2; 29.2, CH2; 29.7, CH2; 29.8, CH2; 29.9, CH2; 30.0, CH2; 32.3, CH2; 124.4, 3-CHBr; 148.1, C3; 195.5, C2. Mass spectrum: m/z 290 (M(⁸¹,Br), 3%); 288 (M(⁷⁹Br), 3%); 275 (10); 273 (10); 209 (100), 191 (40), 165 (30), 151 (90), 135 (50). 111 (100).

### 2-(Bromomethylene)-3-Pentanone (Compound 123)

2-(Bromomethylene)-3-pentanone was prepared by bromination followed by decarboxylation of 4-oxo-3-methyl-2-hexenoic acid as described in the general method.

Yield 58%. vₘₐₓ 3090, 2955, 2910, 1665, 1590, 1450, 1410, 1370,1350, 1280, 1190, 1080, 1040, 980, 930, 770, 720 cm⁻¹. ¹H n.m.r. δ (CDCl₃)1.11, t, J 7.2 Hz, (H5)3; 1.97, s, (H1)3; 2.66, q, J 7.2 Hz, (H4) 2; 7,47, s, 1H, 2-CHBr. ¹³C n.m.r. δ (CDCl₃); 8.2, C5; 15.0, C4; 31.2, Cl; 122.9, 2-CHBr; 142.5, C2; 198.2, C3. Mass spectrum: m/z 178 (M(⁸¹Br), 60%); 176 (M(⁷⁹Br), 60%); 149 (100); 147 (100); 121 (70). 119 (70).

### Example 4

### 4-Bromo-3-Phenylbut-3-En-2-One

To a solution of bromine (1 g, 0.3 ml) in dichloromethane (2 ml) was added drop wise with stirring to an ice-cooled solution of the keto acid (1 g, 5.26 mmol) in dichloromethane (20 ml) containing DBU (0.07 g, 5.79 mmol). The solution was stirred at room temperature for an hour and excess bromine was destroyed by careful addition of a saturated solution of sodium metabisulfite. The organic phase was separated, washed with brine, dried over anhydrous sodium sulfate and evaporated under reduced pressure. The residual viscous oil was triturated with dichloromethane/light petroleum to yield the bromomethylene compound (0.74 g, 62.5%) as a while solid (colourless granules from light petroleum). M.p. 97-100° C. vₘₐₓ (nujol) 3279, 1727, 1450, 137, 1295, 1124, 1087, 89016 777 and 751 cm−1. λₘₐₓ 275 (ε26286), 221 (14377) and 214 (17992) nm. ¹H nmr δ (CDCl3) 7.78-7.80 (m, 2H, ArH); 7.44 (m, 3H, ArH); 6.24 (s, 1H, CH═Br); 1.80 (s, 3H, Me).

### Example 5

### Effect of 3-(Bromomethylene)-2-Butanone (80) and 2-(Bromomethylene)-3-Pentanone (123) as Inhibitor of Two-Component Signal Transduction Systems (Beta Gulactosidase Activity)

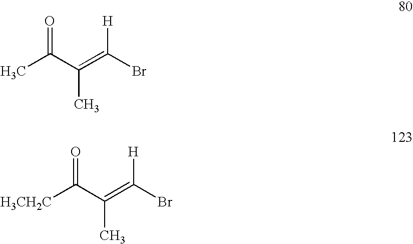

### Two-Component Signal Transduction Assays

### Taz-1 Assay

The Taz-assay carried out according to the method of Jin and Inouyc (1993) with the following alterations. E. coli RU1012 (pYT0301) were grown overnight in M9 medium at 37° C. supplemented with 100 ug/ml ampicillin and 50 ug/ml kanamycin. This overnight culture was then used to inoculate 50 ml M9 medium in side-arm flasks which were then incubated at 37° C. and shaken at 180 rpm. The OD₆₁₀ of the growing cultures was monitored regularly and when the OD₆₁₀=0.2 the cultures were placed on ice to side-arm flasks to give a final concentration of 3 mM (aspartate stock solution made up in M9 salts).

The test compound or mixtures of compounds were dissolved in ethanol and added to cultures to give the required final concentrations. Negative controls were prepared with equal volumes of ethanol. Cultures were then placed in a 37° C. incubator and shaken for 4 hours (OD₆₁₀ approximately 0.7) before being removed and put on ice. Samples were then removed for beta-galactosidase assays carried out according to the method of Miller (1972). Aspartate (the natural inducer of the Taz system) was used as a positive control at a concentration of 3 mmolar.

The results show (see FIGS. 1 and 2) that 3-(bromomethylene)-2-butanone (80) reduced the beta-galactosidase activity by 75% at a concentration of 50 ug/ml,

2-(Bromomethylene)-3-pentanone (123) reduced the activity by 40% at a concentration of 25ug/ml.

### Example 6

### Effect of 3-(Bromomethylene)-2-Hexanone (122) and 2-(Bromomethylene)-3-Pentanone (123) Against Growth of Staphylococcus Aureus

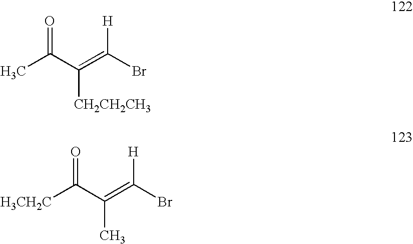

### Methods and Results

Compounds 122 and 123 were tested against growth of Staphylococcus aureus. The experiments were performed in sidearm flasks and the growth was measured at 610 nm for 12 hours. Hundred μl of an overnight culture was added to 10 ml of growth medium. Nutrient Broth, containing furanones at concentrations 10 and 25 μg/ml. The bacteria were incubated at 37° C.

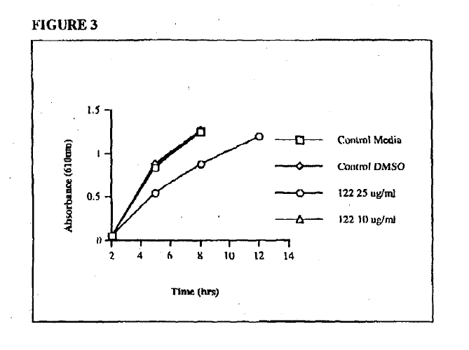

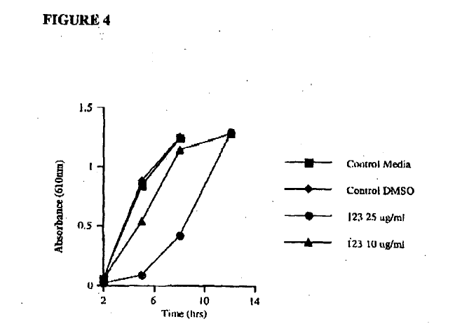

The result (see FIGS. 3 and 4) showed that furanone 123 was more growth inhibitory against S. aureus compared to 122. Twenty-five μg/ml of furanone 123 gave a 4 hours prolong lagphase of growth. A slight growth inhibition could be demonstrated with furanone 122 at 25 μg/ml and furanone 123 at 10 μg/ml.

### Example 7

### Effect of 3-(Bromomethylene)-2-Hexanone (122) and 2-(Bromomethylene)-3-Pentanone (123) Against Attachment of Pseudomonas Aeruginosa (PA01 DO)

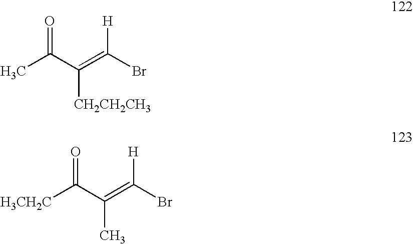

The halomethylene alkanones 122 and 123 were tested for their effect on the attachment of Pseudomonas aeruginosa (PA01 DO) in accordance with the following protocol:

The experiments were performed in 96 wells microtiter plates using a volume of 200 μl. The growth medium was M9 and the plates were incubated at 37° C. One % of overnight inoculum was used and the concentrations of the compounds to be tested were 25 and 50 μg/ml. The attachment of the cells were monitored at the endpoint of the experiment which was after 24 hrs. The cells were stained with crystal violet and the absorbance was measured at 595 nm. Reduction in attachment was measured against the control (PAO1 Do ETO ) set at 100%.

The halomethylene alkanones were found (see FIG. 5) to inhibit the attachment of Pseudomonas aeruginosa (PAO1 DO). For example 3-(bromomethylene)-2-hexanone (122) and 2-(bromomethylene)-3-pentanone (123) reduced the attachment of Pseudomonas aeruginosa (PAO1 DO) by up to 50% at a concentration of 50 ug/ml.

### Example 8

### V. Harveyi Bioassay for the Detection of Al-2 Activity

The V. harveyi bioassay was performed as described previously (Surette and Bassler, 1998). The V. harveyi reporter strain BB170 was grown for 16 hours at 30° C. with shaking in AB medium. Cells were diluted 1:5,000 into 30° C. prewarmed AB medium and 90 ul of the diluted suspension was added to wells containing supernatant. Compounds to be tested were added to the wells to achieve the desired final concentrations and the final volume in each well was adjusted with sterile medium to 100 ul. Ten ul of V. harveyi BB152 (Al-1−, Al-2+) supernatant was used as a positive control and 10 ul of E. coli DH5α supernatant or sterile media was used as a negative control. This strain of E. coli has previously been shown to harbor a mutation in the Al-2 synthase gene, ygαG, which results in a truncated protein with no Al-2 activity (Surette et al. 1998). The microtiter plates were incubated at 30° C. with shaking at 175 rpm. Hourly determinations of the total luminescence were quantified using the chemiluminescent setting on a Wallac (Gaithersburg, Md.) model 1450 Microbeta Plus liquid scintillation counter. The V. harveyi cell density was monitored by the use of a microplate reader (Bio-Rad, Hercules, Calif.). Activity is reported as the percentage of activity obtained from V. harveyi BB152 cell-free supernatant. While the absolute values of luminescence varied considerably between experiments, the pattern of results obtained was reproducible.

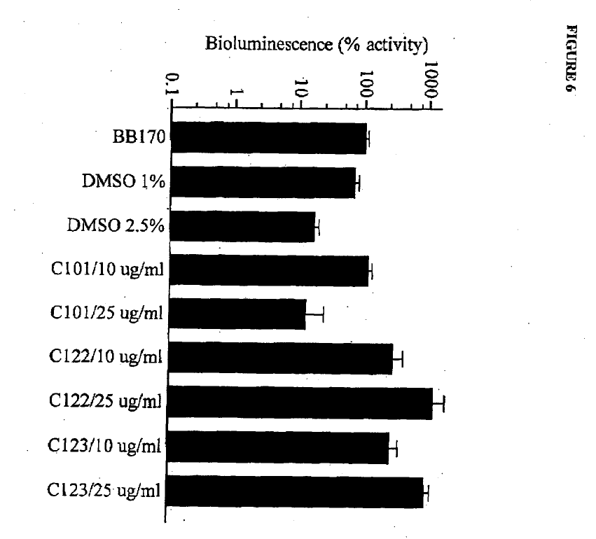

The halomethylene alkanones were found to up regulate the bioluminescence activity in Vibrio harveyi Al-2 assay. For example 3 (bromomethylene)-2-heptanone (101), 3-(bromomethylene)-2-hexanone (122) and 2-(bromomethylene)-3-pentanone (123) caused a significant increase in bioluminescence activity in Vibrio harveyi Al-2 assay at a concentration of 50 ug/ml (see FIG. 6).

### Example 9

### Effect of Compounds 123 and 124 on the Attachment and Growth of Porphyromonas Canoris

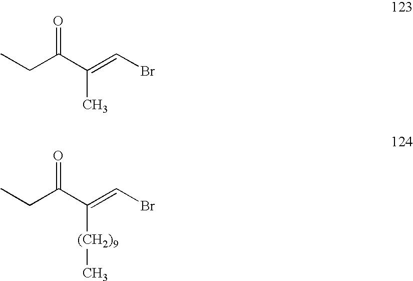

The effect of compounds 123 and 124 on the growth and attachment of the bacteria Porphyromonas canoris was determined using the following protocol:

The experiments were performed in 96 well microtiter plates using a volume of 100 μl. The growth medium was BHI and the plates were incubated at 37° C. One % of overnight inoculum was used and the concentration of the compound to be tested was 50 μg/ml. Both growth and attachment of the cells were monitored at the end point of the experiments which was and after 24 hrs with P. canoris. The cells were stained with crystal violet and the absorbance was measured at 595 nm.

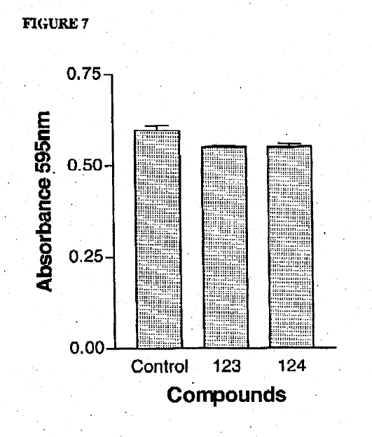

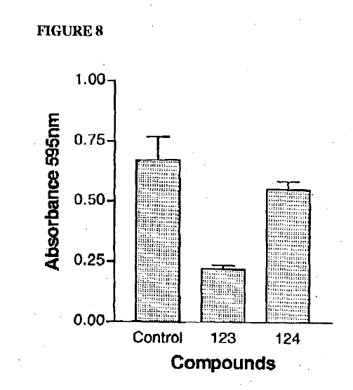

FIGS. 7 and 8 show the effects of each compound on growth and attachment respectively.

### Example 10

### Effect of Compounds 123 on the Attachment and Growth of Pseudomonas Aeruginosa

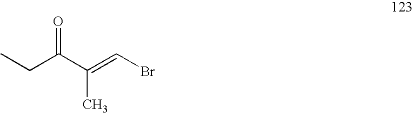

The effect of compounds 123 on the growth and attachment of the bacteria Pseudomonas aeruginosa was determined using the following protocol:

As Example 9, but the used medium was a 1:9 dilution of TBY medium and the incubation time was 6 hrs.

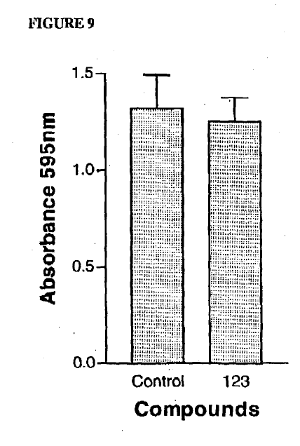

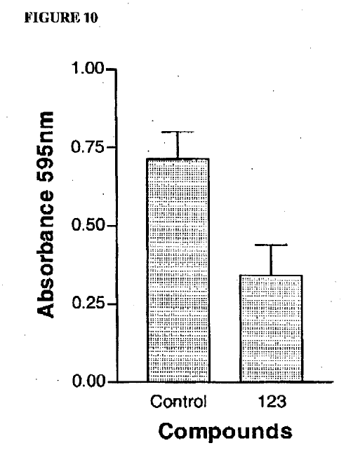

FIGS. 9 and 10 show the effects of compound 124 on growth and attachment respectively.

Throughout this specification the word “comprise”, or variations such as “comprises” or “comprising”, will be understood to imply the inclusion of a stated clement, integer or step, or group of elements, integers or steps, but not the exclusion of any other clement, integer or step, or group of elements, integers or steps.

All publications mentioned in this specification are herein incorporated by reference. Any discussion of documents, acts, materials, devices, articles or the like which has been included i in the present specification is solely for the purpose of providing a context for the present invention. It is not to be taken as an admission that any or all of these matters form part of the prior art base or were common general knowledge in the field relevant to the present invention as it existed anywhere before the priority date of each claim of this application.

It will be appreciated by persons skilled in the art that numerous variations and/or i modifications may be made to the invention as shown in the specific embodiments without departing from the spirit or scope of the invention as broadly described. The present embodiments are, therefore, to be considered in all respects as illustrative and not restrictive.

### REFERENCES

Fenical, W and Mconnell, O. J. Antibiotics and antiseptics compounds from die family Bonnemaisoniaceae, Proc. Int. Seaweed. Sym., 1979, 9, 387-400.

Jin, T., and M. Inouye. 1993. Ligand binding to the receptor domain regulates the ratio of kinase to phosphatase activities of the signalling domain of the hybrid Escherichia coli transmembrane receptor, Taz1. J. Mol. Biol. 232: 484-49

McConnell, O. J., Fenical, W. Polyhalogenated 1-octen-3-oues, antibacterial metabolites from the red seaweed Bonnemaisonia Asparagoides, Tetrahedron Letts., 1977, 1851-1854.

Miller, J. H. 1972. Experiments in molecular genetics. Cold Spring Harbor Laboratory, Cold Spring Harbor,. N.Y.

Surette, M. G, and B. L. Bassler. 1998. Quorum sensing in Escherichia coli and Salmonella typhimurium, Proc. Natl. Acad. Sci., USA 95:7046-7050.

Surette, M. G., M. B. Miller, and B. L. Bassler. 1999. Quorum sensing in Escherichia coli, Salmonella typhimurium, and Vibrio harveyi: a new family of genes responsible for autoinducer production. Proc. Natl. Acad. Sci., USA 96:1639-1644.

Wei, Han-Xun, Kim. S. H., Caputo, T. D., Purkiss, D. W. and Li, G., Highly stereoselective alpha-hydroxylation/chlorination of alpha,beta-acetylenic ketones—An efficient approach to beta-halegeno Baylis-Hillman adducts, Tetrahedron, 2000, 56, 2397-2401.

1. A method for the preparation of a compound of formula II 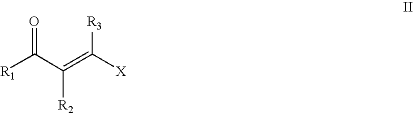
wherein R₁, R₂ and R₃, which may be the same or different, are independently selected from H, halogen, alkyl, alkoxy, alkenyl, alkynyl, aryl, arylalkyl, carboxyl, acyl, acyloxy, acylamino, formyl and cyano whether unsubstituted or substituted, optionally interrupted by one or more hetero atoms, straight chain or branched chain and X is a halogen;
the method comprising decarboxylating a compound of formula I 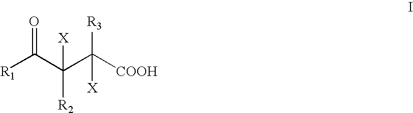
wherein R₁, R₂, R₃ and X are as defined above, and
wherein the decarboxylation is carried out in the presence of a mild base, optionally in the presence of a solvent.

2. A method according to claim 1 wherein:
R₁, R₂ and R₃, which may be the same or different, are independently selected from H, halogen, alkyl, alkoxy, oxoalkyl, alkenyl, aryl or arylalkyl whether unsubstituted or substituted, optionally interrupted by one or more hetero atoms, straight chain or branched chain;
X is a halogen;

3. A method according to claim 1 wherein:
R₁, R₂ and R₃ are independently H, halogen, alkyl, alkoxy, oxoalkyl, alkenyl, aryl or arylalkyl whether unsubstituted or substituted, optionally interrupted by one or more hetero atoms, straight chain or branched chain; and
X is Br, F or I;

4. A method according to claim 1 wherein:
R₁, R₂ and R₃, which may be the same or different, are independently selected from H, halogen, alkyl, alkoxy, oxoalkyl, alkenyl, aryl or arylalkyl whether unsubstituted or substituted, optionally interrupted by one or more hetero atoms, straight chain or branched chain; and
X is a Br.

5. A method according to claim 1 wherein at least one of R₁, R₂, R₃ is an alkyl group.

6. A method according to claim 5 wherein at least one of R₁ and R₂ is alkyl and R₃ is H.

7. A method according to claim 1 wherein X is Br.

8. A method according to claim 1 wherein the mild base is selected from triethylamine or DBU, optionally in the presence of other bases.

9. A method according to claim 1 wherein the mild base is triethylamine.

10. A method according to claim 9 wherein the solvent is selected from the group consisting of dichloromethane, dichloroethane and trichloroethane.

11. A compound of formula II produced by the method according to claim 1.

12. A compound of formula II: 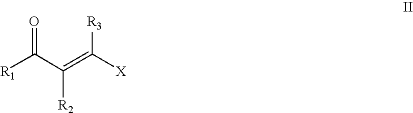
wherein:
R₁ is alkyl;
R₂ is alkyl or aryl;
R₃ is H;
X is Br or F.

13. A compound according to claim 12 with the proviso that where R1 is methyl R₂ is not C₁₀alkyl, methyl, CH₂CH₂CO₂CH₃, CH₂CH₂NO₂ or CH₂CH₂CH₂OC(O)Ph.

14. A compound according to claim 12 wherein R₁ is C₂₋₁₀ alkyl.

15. A compound according to claim 14 wherein R₁ is ethyl.

16. A compound according to claim 12 wherein X is Br.

17. The compound 2-(bromomethylene)-3-pentanone.

18. Use of a compound of formula II according to claim 12 in a product selected from the group consisting of cleaning agent for use in the home or industrial settings; antifouling paint, water treatment products; antibacterial agents in the treatment of mammals; antibacterial additive or preservative in a medical or surgical device, disinfectant, soap formulation, shampoo formulation, hand wash formulation, dentrification formulation, detergent for laundry or dishes, wash and treatment solution for topical use; and contact lenses.

19. A compound according to claim 13 wherein R₁ is C₂₋₁₀ alkyl.
